(19) United States
(12) Patent Application Publication
(10) Pub. No.: US 2017/0322211 A1
(43) Pub. Date: Nov. 9, 2017

(54) PATHOGEN DETECTION BY SERS NANOPARTICLES

(71) Applicant: Colorado School of Mines, Golden, CO (US)

(72) Inventors: Nicholas R. Stambach, Golden, CO (US); Stephanie A. Carr, Golden, CO (US); Christopher R. Cox, Golden, CO (US); Kent J. Voorhees, Golden, CO (US)

(21) Appl. No.: 15/497,836

(22) Filed: Apr. 26, 2017

Related U.S. Application Data
(60) Provisional application No. 62/327,812, filed on Apr. 26, 2016.

(51) Int. Cl. G01N 33/569; G01N 21/65; G01N 33/532; G01N 33/553; C07K 16/08; G01N 33/543

(52) U.S. Cl. CPC G01N 33/56983; G01N 33/54393; G01N 21/658; G01N 33/56911; G01N 33/553; C07K 16/081; G01N 33/54346; G01N 33/532; G01N 2333/01; G01N 2333/195

(57) ABSTRACT

A phage specific antibody presenting particle, devices and methods related to detection of phage amplification are provided. Specifically, described herein are compositions and methods for detecting Listeria bacteria using an antibody that recognized A511 anti-Listeria phage, wherein the antibody is conjugated to a nanoparticle that can be detected by surface-enhanced Raman scattering (SERS).

### CROSS-REFERENCE TO RELATED APPLICATIONS

This application claims benefit of priority pursuant to 35 U.S.C. §119(e) of U.S. provisional patent application No. 62/327,812 entitled “Pathogen Detection By SERS Nanoparticles,” filed on Apr. 26, 2016, which is hereby incorporated by reference in its entirety.

### FIELD

The disclosed processes, methods, and systems are directed to rapid, economical, selective, and sensitive identification of pathogens, especially bacteria.

### BACKGROUND

Rapid and accurate detection of bacteria and other pathogens is critical for many industries, including the food and agriculture industries. However, many existing methods are slow, complex, expensive, and/or lack the ability to differentiate between live and dead organisms. For example, genotypic identification methods (for example PCR-based techniques) are very accurate, but require expensive and slow DNA extraction, amplification, and separation/sequencing protocols. In addition, genotypic identification cannot differentiate live from dead microorganisms.

Listeriosis is of particular concern for food and agriculture industries. Specifically, Listeriosis is responsible for approximately about 1600 food-related illnesses; 94% of which result in hospitalization. Listeriosis is also known to contribute to spontaneous abortions and it has a mortality rate of 20-30%. The causative agent in listeriosis is the bacteria Listeria monocytogenes.

Despite the statistics provided above, conventional methods of detecting L. monocytogenes are too slow to analyze the majority of food products before they are purchased, handled, or eaten by a food worker or consumer. Because Listeria is easily transmitted by contact with soil and animals, there is a substantial risk that processed foods may contain Listeria. Moreover, Listeria's ability to grow at temperatures as low as 1° C., means that refrigeration may be ineffective at preventing its spread.

What is needed is a rapid, economical system for detecting pathogens that is the sensitive and selective and can be performed in the absence of complex, costly lab equipment and personnel. This and other needs are addressed by the present disclosure.

### SUMMARY

The present disclosure provides compositions and methods for rapid and reliable detection of one or more pathogens. In one embodiment, a phage-specific antibody presenting nanoparticle is described. In another embodiment, a method of making a phage-specific antibody presenting nanoparticle is described.

While multiple embodiments are disclosed, still other embodiments of the invention will become apparent to those skilled in the art from the following Detailed Description, which shows and describes illustrative embodiments of the invention. As will be realized, the invention is capable of modifications in various aspects, all without departing from the spirit and scope of the invention. Accordingly, the drawings and detailed description are to be regarded as illustrative in nature and not restrictive.

### BRIEF DESCRIPTION OF THE DRAWINGS

FIG. 1A is a schematic representation of an LFI device.

FIG. 1B is a graph of the Raman spectrum for trans-1,2-bis(4-pyridyl)-ethylene, SERS NP S440.

FIG. 1C is a schematic representation of an LFI device.

FIGS. 2A-2E depicts LFI devices used for determining visual LOD—as determined by the presence or absence of a pink line at the test line. FIG. 2A) 1.46×10⁹ pfu mL⁻¹, visual+, SERS+; FIG. 2B) 1.78×10⁸ pfu mL⁻¹, visual+, SERS+; FIG. 2C) 6.10×10⁷ pfu mL⁻¹, visual+, SERS+; FIG. 2D) 6.37×10⁶ pfu mL⁻¹, visual−, SERS+; FIG. 2E) negative control, visual−, SERS−.

FIG. 3 depicts a graph showing the determination of limit of detection using serial dilutions of phage. Phage concentration vs. Raman intensity for dilution of phage. The SERS LOD is indicated by a dashed line.

FIGS. 4A and 4B are graphs of SERS intensity (FIG. 4A) and phage concentration (FIG. 4B) as a function of time. Four phage amplification experiments were performed at MOI 0.1 and assayed using SERS-LFI (4A) and parallel spot titer assay (4B).

FIGS. 5A and 5B are graphs showing SERS intensity (FIG. 5A) and phage concentration as a function of time. Amplification was carried out at 3 different MOIs utilizing a constant starting phage concentration of 5×10⁵ pfu mL⁻¹.

### DETAILED DESCRIPTION

The present disclosure provides a phage specific antibody presenting particle, devices and methods related to detection of pathogens. In particular, the disclosed devices and methods are able to quickly and accurately detect the presence of live Listeria monocytogenes. In some embodiments, detection of Listeria monocytogenes is accomplished by detecting phage that are specific for Listeria monocytogenes. The detection of Listeria-specific phage may aid in reducing false positives due to dead or inactive Listeria monocytogenes. In addition, detection of Listeria-specific phage may help enhance the sensitivity of the assay. The disclosed methods and devices may be used in detecting the presence of a variety of target pathogens and microorganism, such as a bacteria or fungi, in a sample. In various embodiments, the disclosed methods and devices may be used to identify two or more different pathogens in a sample. The disclosed devices and methods provide for enhanced specificity, sensitivity, simplicity, speed, and cost effectiveness of detecting micro-organisms.

Listeria monocytogenes is a Gram-positive, motile, facultative anaerobic rod and the etiological agent of food-borne listeriosis. Symptoms of listeriosis include gastroenteritis, diarrhea, meningitis and bacteremia. It also contributes significantly to spontaneous abortions. Listeriosis is responsible for approximately 1600 food-related illnesses and 260 deaths in the U.S. annually and is the third leading cause of death among foodborne pathogens (behind Salmonella spp. and Toxoplasma gondii), with 94% of cases leading to hospitalization and 20-30% resulting in death.

For cooked food, both the USDA (United States Department of Agriculture) and the FDA (Food and Drug Administration) have adopted a zero tolerance policy for Listeria—that is, there can be no detectable Listeria monocytogenes. However, because of the protracted turnaround times (TAT) of conventional detection methods, the majority of food products potentially contaminated with L. monocytogenes are not tested before entering the marketplace. This delay requires manufacturers to issue recalls of the food after an outbreak has been detected, thus increasing the risk that limited outbreaks will become widespread. Moreover, recalls are expensive and generally not completely effective.

Given Listeria's natural occurrence in soil and among animal reservoirs, many foods are at risk for Listeria contamination. These include ready-to-eat meats and cheeses, unpasteurized dairy products, hot dogs, smoked seafood, as well as raw meat and produce. Further adding to difficulties in outbreak prevention is Listeria's ability to grow over a wide range of temperatures, including those as low as 1° C., which allows it to propagate even when refrigerated (˜4° C.). One example of this is a 2011 outbreak associated with contaminated whole cantaloupes from Colorado. 147 people were infected across 28 states resulting in 33 deaths and one miscarriage, making it the second deadliest foodborne related outbreak in U.S. history.

Phage are predatory bacterial viruses that have long been utilized for bacterial detection and identification and are simple and inexpensive to produce. Briefly, phage amplification involves phage attachment to the surface of a host bacterium, followed by insertion of its nucleic acid. This results in hijacking of the host replication machinery and subsequent transcription and translation of phage genes. Replication and assembly of progeny phages follows, culminating in host cell lysis and release of new infectious virions (progeny phage) into the surrounding milieu to repeat the cycle. This leads to a rapid increase (termed burst) in phage concentration in less than 3 h. Analytical techniques, such as matrix-assisted laser desorption/ionization time-of-flight mass spectrometry can be used to detect progeny phage, which act as secondary biomarkers for species-specific bacterial detection.

For the reasons above, Listeria is an excellent test subject for investigating the effectiveness of the disclosed methods, devices, and systems. Disclosed herein is a method of identifying Listeria through identification of phage particles specific for that bacteria. Specifically, detection of Listeria phage A511 is disclosed. Phage A511, is a large (134,494 kbp), well-characterized lytic myovirus with a broad-host range. Phage A511 has the ability to produce many progeny phage (burst size 40-50 phage) in a short amount of time—latent period 55-60 min at 30° C.

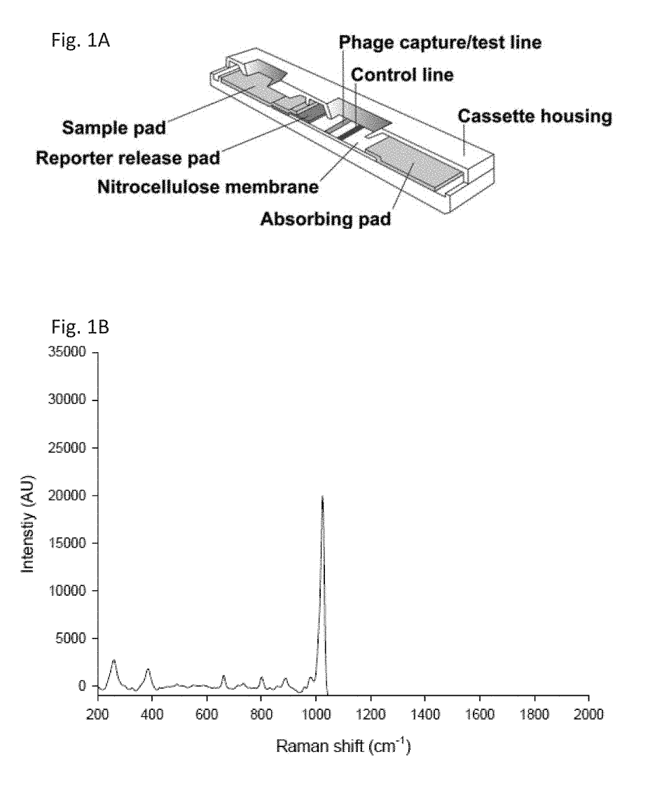

The use of Lateral Flow Immunochromatography (LFI), is disclosed, to identify bacteria and other micro-organisms using a SERS, phage-based detection system. In most cases, LFI is a chromatographic technique wherein a liquid sample is applied to a portion of a paper embedded with detector molecules. FIG. 1A shows one embodiment of an LFI device. An analyte in the sample is transported by capillary action across a paper or membrane. As the analyte diffuses from one end to the to the other, it enters a region of the paper referred to as a “release pad” or “reagent area” where reporter particles conjugated with analyte-specific antibodies bind to the analyte. Various reporter particles can be used, including colored, fluorescent, spectroscopic, or enzymatically labeled particles. Once bound, reporter-analyte complexes continue to flow along the membrane until reaching a “capture zone” or “target zone” composed of immobilized analyte-binding molecules (again, typically antibodies). By interacting with the immobilized analyte-binding molecules, the reporter complex is concentrated. The concentrated reporter molecule thus becomes detectable—visible or otherwise—at a test line.

In most cases, the LFI also includes control particles. The control particles flow with the analyte-specific complexes across the membrane. The control particles then bind a molecule immobilized at a second line, downstream from the test line. When the control line becomes visible or detectable, the end user can be assured that the LFI worked properly—transporting reagents through the test line.

The devices and methods disclosed herein comprise a SERS nanoparticle having a gold core and an organic, Raman-active reporter dye (for example trans-1,2-bis(4-pyridyl)-ethylene; see FIG. 1B). In many embodiments of the disclosed nanoparticle, the silica surface is functionalized with thiol groups to aid in the attachment of anti-phage antibodies.

At high concentrations SERS particles can be detected by eye in LFI. However, at low analyte concentrations, test lines may be too faint to detect by eye. The use of Raman spectroscopy may extend sensitivity of the detection assay below visual levels and provide a quantifiable signal, thus eliminating the need for visual conformation and providing the possibility that analyte concentrations may be determined.

The disclosed ability to detect pathogenic (and other) micro-organisms in a simple, efficient, sensitive and cost-effective manner is advantageous for many applications, including, but not limited to, human clinical and field diagnostics, environmental testing, food pathogen detection, veterinary diagnostics, and bio-warfare detection. As disclosed herein, amplification of a bacteria-specific phage and detection of the amplified bacteria-specific phage may allow identification of low levels of bacteria, along with the ability to distinguish live, rather than dead bacteria, in a sample. Lateral flow devices and techniques, combined with SERS, may provide for detection limits at or below approximately 20 fM or 1×10⁶ PFU (plaque forming units).

### Nanoparticles

In one embodiment of a nanoparticle for use with the disclosed devices and methods, the nanoparticle may display a phage specific antibody on its outer surface. The nanoparticle may include a metal core and a reporter molecule surrounding or the core or positioned on or at the surface of the core. A coating surrounds the core and reporter molecule. Electromagnetic waves (such as visible and invisible light waves) can pass through the coating. The coating may also aid in attaching a phage-specific molecule, such as an antibody, to the nanoparticle. In most embodiments, the nanoparticle has a diameter D_{NP}, which is between approximately 21 nm and approximately 240 nm, for example about 120 nm.

The metal core can be made of any metal that provides for enhanced Raman scattering. The metal is selected from gold (Au), silver (Ag), copper (Cu), sodium (Na), potassium (K), chromium (Cr), aluminum (Al), lithium (Li), or a combination or alloy thereof. In some embodiments, the metal core can be pure metal or a metal alloy and can be overlaid with at least one metal shell, such as a core-shell particle comprised of Au/AuS. In a preferred embodiment, the core is comprised of gold.

The metal shell can be chosen so as to maximize the Raman signal's intensity from the reporter molecule coating. The metal core can have any shape, including a sphere, ellipsoid, or cylinder. In cases where the metal core is a sphere, its diameter, D_{C}, can be between approximately 15 nm and approximately 200 nm. In some embodiments, the diameter of the core may be between approximately 40 nm and approximately 100 nm. In one embodiment, the metal core has a diameter of about 60 nm. In various embodiments, the core can have a diameter of greater than 10 nm, 20 nm, 30 nm, 40 nm, 50 nm, 60 nm, 70 nm, or 80 nm, and/or less than 300 nm, 280 nm, 260 nm, 240 nm, 220 nm, 200 nm, 190 nm, 180 nm, 170 nm, 160 nm, 150 nm, 140 nm, 130 nm, 120 nm, 110 nm, or 100 nm. Where the metal core is other than spherical in shape, at least one dimension of the core is as described for the spherical embodiments above.

### Antibodies

For purposes of the present disclosure, the term “antibody” is used in the broadest sense and specifically covers monoclonal antibodies, polyclonal antibodies, multispecific antibodies, e.g., bispecific antibodies, chimeric antibodies, humanized antibodies, fully synthetic antibodies, single domain antibodies, and antibody fragments so long as they exhibit the desired biologic activity, i.e., binding specificity. An antibody is a monomeric or multimeric protein comprising one or more polypeptide chains. An antibody binds specifically to an antigen and can be able to modulate the biological activity of the antigen. The term “antibody” also includes antibody fragments. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546) which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883), (viii) bispecific single chain Fv dimers (PCT/US92/09965) and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448). In certain embodiments, antibodies are produced by recombinant DNA techniques. Other examples of antibody formats and architectures are described in Holliger & Hudson, 2006, Nature Biotechnology 23(9):1126-1136, and Carter 2006, Nature Reviews Immunology 6:343-357 and references cited therein, all expressly incorporated by reference. In additional embodiments, antibodies are produced by enzymatic or chemical cleavage of naturally occurring antibodies.

### Phage

In this disclosure, the term “phage” include bacteriophage, phage, mycobacteriophage (such as for TB and paraTB), mycophage (such as for fungi), mycoplasma phage or mycoplasmal phage, and any other term that refers to a virus that can invade living bacteria, fungi, mycoplasmas, protozoa, and other microscopic living organisms and uses the host organism to replicate itself. Note that for purposes of this disclosure any phage that can specifically adsorb with a microorganism can be useful, however, for simplicity sake, the disclosure often refers to bacteriophage and bacterium.

Bacteriophage are viruses that use, for example, bacteria as a way to replicate themselves. Other phages use other organisms, such as fungi. A bacteriophage does this by attaching itself to a bacterium and injecting its DNA into that bacterium, inducing it to replicate the bacteriophage hundreds or even thousands of times. Some bacteriophage, called lytic bacteriophage, rupture the host bacterium, releasing the progeny phage into the environment to seek out other bacteria. The total incubation time for phage infection of a bacterium, phage multiplication or amplification in the bacterium, to lysing of the bacterium may take anywhere from minutes to hours, depending on the bacteriophage and bacterium in question and the environmental conditions.

Detection of phage amplification can be performed in various ways. In one embodiment, a parent phage is amplified by infecting a bacterium, replicating, and bursting the bacterium to create progeny phage. Progeny phage can then be exposed to one or more of the disclosed particles, and the particle detected by Raman spectroscopy—thus detecting the progeny phage.

Generally, a phage that is specific to a target microorganism is introduced into a sample. The phage should infect and multiply within the target microorganism. Conditions are provided such that the phage is allowed to infect the target microorganism and multiply in the target to create a detectable amount of phage or biological substance associated with the bacteriophage in the sample. The microorganism is then lysed. Lysis of the microorganism may result from multiplication of the phage or by enzymatic, chemical, or other lysing methods, for example, addition of a bacterial lysozyme enzyme, where the target is a bacterium.

The disclosed devices, nanoparticles, and SERS methods may be used to detect a parent phage or progeny of the parent phage. For example, parent phage or progeny phage can be recognized and immobilized by the disclosed anti-phage particle to create a particle-phage-antibody-conjugate. In some embodiments, the nanoparticles may comprise an antibody that can bind only the progeny phage and/or different particles can be used for the progeny phage and the parent phage.

### Sample

A sample is assayed for the presence of a microorganism by detecting a microorganism-specific phage or other biological substance. In most embodiments, detection is performed by lateral flow strip and Raman spectroscopy, preferably SERS, as described in more detail below. Where the number of progeny phage increases, this indicates the presence of a live microorganism for which the phage is specific.

A sample comprising one or more target micro-organisms is tested for the presence of the micro-organism. Typically, the sample is provided in a liquid. However, one or more liquids may be added to a dry substance (that may contain the micro-organism) to create a sample. The sample may be pretreated prior to testing/analysis/detection. In some embodiments, the sample is purified, filtered, concentrated, or otherwise processed to remove unwanted components or to enhance the concentration of microorganisms in the sample. A media may be added to the sample to aid in growth of the microorganism and/or a phage that is specific for the microorganism. The sample may include one or more of a sputum sample, blood sample, waste sample, water sample, food sample, surface wipe, filter sample, etc. It should be clear to one skilled in the art that pretest sample preparation can include any suitable process and the raw sample can take many different forms.

A phage may be added to the sample. In some embodiments, the phage may be added in a dry state, or the phage may be mixed or suspended in a vial to which the raw sample is added. The phage added to the raw sample is herein referred to as the “parent phage”.

A test sample comprising the raw sample and the parent phage is incubated and the parent phage infects the target bacteria by attaching themselves to cell walls of the target bacteria and injecting the viral nucleic acid to create infected bacteria. Replication of progeny phage proceeds within the host bacteria. If lytic phage are used, the host will rupture, thereby releasing progeny phage into the test sample. The progeny phage can then infect other target bacteria. If there were target bacteria in the raw sample, the test sample will contain a large number of progeny phage. An additional step can be performed whereby a suitable lysozyme is added to lyse any infected target bacteria that did not lyse naturally. The suitable lysozyme will rupture the cell walls and any progeny phage still held within the host bacteria will be released into the test sample and can now be detected.

### Methods of Detecting Microorganisms

In accordance with the present disclosure, in one embodiment, progeny phage may be contacted with an antibody specific to the progeny phage. The antibody may be attached or conjugated to the surface of a particle, to create a phage-specific antibody-presenting particle. In some embodiments, the phage specific antibody may also be immobilized on a solid surface, for example, a lateral flow strip or other stationary phase. The immobilized antibody may aid in concentrating the phage-specific antibody-presenting particle as it flows across the immobilized antibody. In some embodiments the immobilized antibody may be the same or different than the antibody on the particle. When the phage-specific antibody-presenting particle contacts a phage, a particle-phage conjugate is created. The particle-phage conjugate may then interact with the antibodies immobilized on the solid surface or other stationary phase. The solid surface or stationary phase may include a lateral flow strip, lateral flow chromatography, other lateral flow device, a solid column, and magnetic beads (i.e. where antibodies are attached to a glass-coated magnet). When the phage-particle conjugate is captured by the immobilized antibody, a particle-phage-antibody conjugate is created.

The particle-phage-antibody conjugate can be detected based on a characteristic Raman spectrum provided by the reporter molecule coating of the particle. The concentration of the phage in the incubated test sample is higher than that of the target bacteria in the raw sample because of phage amplification. Thus, lower concentrations of bacteria can be detected where the method, device, kit or process is detecting amplified phage.

Where the particle-phage-antibody conjugate is detected by lateral flow immunochromatography (LFI), the LFI device may comprise an LFI strip having a sample application pad and a reagent pad located at a first end of the device, and an absorption pad located at a second end of the LFI strip. A flow zone is located between the first and second ends of the LFI strip, along with a capture surface having immobilized phage-specific antibodies. Briefly, the lateral flow device allows a solution comprising progeny phage to diffuse from the sample application pad to the absorption pad along the flow zone. As the sample diffuses through the flow zone, it will pass through the reagent pad where it will interact with the phage-specific antibody presenting particle conjugates. If progeny phage exist in the sample, they will be bound by the phage-specific antibody presenting particle conjugates, which will continue to flow across the strip toward the capture surface where the immobilized phage-specific antibodies will capture, immobilize, and concentrate the phage-specific antibody presenting particle conjugates.

In some embodiments, the phage may already be bound to the particle prior to introduction to the sample application pad. In such a case, the particle-phage conjugate in the sample passes across the capture surface where the phage-specific antibody presenting particle conjugates is immobilized and concentrated. It can be appreciated that other methods resulting in a particle-phage-antibody conjugate are contemplated by and envisioned to be within the scope of the disclosure. Diffusion of the sample from the sample application pad to the capture surface typically takes from two to ten minutes, more preferably from two to six minutes and most preferably from about two to five minutes. The particle of the particle-phage-antibody conjugate is then detected by a Raman spectrometer. As discussed previously, in alternative embodiments, the particle can be detected by any other monochromator or spectrometer or any other optical detection methods known in the art.

The Raman spectrum produced is specific to the type of reporter molecule. Thus, the success of amplification and quantity of progeny phage can be detected based on the presence and/or strength of the Raman spectrum. Note also that one or more wash steps can be incorporated into the assay to facilitate removal of non-bound progeny phage and thereby lower the level of non-specific binding or background within each sample run.

### Reporter Molecules

Because different reporter molecule coatings produce different spectra, the system can be multiplexed. That is, a particle with a specific reporter molecule coating can be designed and used, and detected at the same time. In some embodiments, different reporter molecules are used for different phage, thus enabling a multiplexed assay with several different phages that is able to detected different bacteria.

The reporter molecule can be positioned at or near a metal in the core that enhances Raman scattering from the reporter. In many embodiments, the reporter molecule defines a coating attached to the surface of the metal core. The reporter molecule is a spectroscopically-active molecule that exhibits a simple Raman spectrum. The Raman spectrum of the reporter molecule is enhanced when in close proximity to a metal, such as the surface of the metal core. The diameter D_{O} of the reporter molecule coating is determined by the difference between the diameter D_{G} of the glass coating and the diameter D_{M} of the metal core. A person skilled in the art will recognize that the reporter molecule coating can be any type of molecule with a measurable SERS spectrum, and can be a single layer or multi-layered. However, a reporter molecule coating without measurable Raman activity can also be used. A measurable spectrum is one in which the presence of the reporter molecule coating, and/or possibly the core, can be detected and recognized as a characteristic of the particular reporter molecule coating. Generally, suitable Raman-active reporter molecule coatings have (i) strong Raman activity thus minimizing the number of particles necessary to provide a detectable signal and (ii) a simple Raman spectrum which permits the use of multiple different particles which can be distinguished even if used simultaneously.

The reporter molecule coating can comprise a polymer to which a single or multiple Raman-active molecules are attached. Alternatively, the reporter molecule coating can be a single type of reporter molecule or many different types of reporter molecules. Thus, reporter molecule coatings can include one or more molecular species (positively or negatively charged, neutral or amphoteric) or a non-molecular species such as a negatively or positively charged ion. Non-limiting examples of possible Raman-active molecules number in the millions, and include HCl, Hg, CN⁻, dimethylformamide, diamond, charcoal, polypyrrole, oligonucleotides, rust, sulfur, carbon, citric acid and polyacrylamide. Additionally or alternatively, the reporter molecule coating can include a metal oxide.

In some embodiments, the analyte-specific particle is a SERS nanoparticle, to which antibodies capable of binding the target analytes are attached as described in U.S. patent application Ser. No. 12/351,522, filed Jan. 9, 2009, which is incorporated by reference herein in its entirety.

Surface enhancement can increase Raman signals by three or more orders of magnitude, making it a viable probe for target analytes present at low concentrations. The Raman spectrum of the reporter molecule is enhanced when the Raman-active reporter molecule coating is in close proximity to a metal surface such as the metal core of the disclosed analyte-specific nanoparticle. A person skilled in the art will recognize that the reporter molecule coating can be any type of molecule with a measurable SERS spectrum, for example an organic Raman active molecule, and can be a single layer or multi-layered. A measurable spectrum is one in which the presence of the reporter molecule coating, and/or possibly the core, can be detected and recognized as a characteristic of the particular reporter molecule coating. Generally, suitable Raman-active organic reporter molecule coatings have (i) strong Raman activity thus minimizing the number of particles necessary to provide a detectable signal and (ii) a simple Raman spectrum which permits the use of multiple different particles which can be distinguished even if used simultaneously.

Raman-active dyes produce a Raman spectrum, and different dyes typically have different spectra. These unique spectra can aid in identification and signal quantification. Moreover, because the spectra are unique, multiple particles can be used with each particle producing a unique Raman spectrum. This makes possible simultaneous detection of multiple target analytes.

### Coatings

The coating surrounding the metal core and reporter molecule can be selected from various compounds. In one embodiment, the coating is comprised of SiOₓ, and can be modified for operably attaching phage-specific molecules. The coating also provides a barrier to protect or otherwise keep the surrounding solvent away from the reporter molecule and metal core.

Preferably, the glass coating will not have a measurable effect on the SERS activity. However, if the glass coating does have a measurable effect, it will not interfere with the SERS activity. The thickness of the glass coating can vary, depending on its effect on the intensity of the reporter molecule's Raman spectrum. That is, if the glass coating is too thick, it may be difficult to obtain an intense Raman spectrum. In various embodiments, reporter molecules may be selected, and SERS may be performed, as described, for example in Haynes et al., Surface Enhanced Raman Spectroscopy, Anal. Chem. 339A-346A (2005), incorporated herein by reference in its entirety.

### Detectors and Detection

The sensitivity of detecting SERS particles is enhanced by the use of spectrometry. Thus, a detector for use with the disclosed methods and devices can be a spectrometer. When the reporter molecule is a Raman-active reporter molecule in proximity to a metal surface, the reporter molecule coating exhibits an enhanced Raman spectrum. An immobilized phage antibody particle conjugate can thus be detected by Raman spectrometry, or a detector can be a Raman spectrometer. In some embodiments, the Raman spectrometer is a benchtop model. In other embodiments, the Raman spectrometer is handheld.

The peaks in a single Raman spectrum are typically distinct and easily resolvable for each reporter molecule coating. While Raman spectrometers are used in various embodiments, any form of monochromator or spectrometer that can temporally or spatially resolve photons. Any type of photon detector known in the art can be used.

Detection can be performed using visible or near-IR irradiation. Other optical detection/interrogation methods known in the art, and which can be utilized exclusively or in combination with SERS as described in the present disclosure, include: use of a resonantly-excited reporter molecule coating or surface enhanced resonance Raman scattering (SERRS), surfaced enhanced infrared absorption spectra (SEIRA), surface enhanced hyper Raman scattering (SEHRS), as well as the resonant analogue SEHRRS.

The Raman spectrometer can be a commercially available Raman spectrometer. Examples of commercially available Raman spectrometers include, but are not limited to, Raman spectrometers from the following companies: DeltaNu (Laramie, Wyo.), Thermo, Rigaku, Perkin Elmer, Ocean Optics, Bruker, Enwave, and Lambda Solutions.

While Raman spectrometers are used in various embodiments, any form of monochromator or spectrometer that can temporally or spatially resolve photons and any type of photon detector known in the art can be used.

### Examples

### Experiment I—SERS-LFI Device Optimization

Disclosed herein are experiments designed to aid in optimization of SERS-LFI for phage detection, specifically: antibody-nanoparticle conjugation; the composition of the LFI running buffer composition; antibody:SERS NP ratio; blocking of non-specific phage-particle interactions; capture antibody application; membrane flow rate; and SERS reporter release pad concentration. Troubleshooting of these parameters has helped develop a new, robust protocol for fabrication and use of SERS-LFI devices.

Optimization and standardization of antibody conjugation to SERS NPs is intended to enhance reproducibility and consistency of the disclosed methods. Conjugation optimization was performed to help minimize agglomeration of conjugated NPs on the membrane that was experienced with prior methods. Agglomerated NPs travel down the nitrocellulose very inefficiently, creating artificially high false-positive signals. Agglomeration was even seen in controls that were analyte-free.

### Production and Purification of Anti-A511 Antibodies

Polyclonal rabbit anti-A511 phage IgG was prepared by Antibodies Incorporated (Davis, Calif., USA). Antibodies were Protein G purified (Nab™, Thermo Scientific, Rockford, Ill., USA) and specificity was confirmed by enzyme-linked immunosorbent assay (ELISA). Purified antibodies were dialyzed in PBS, concentrated by ultrafiltration (Amicon® Ultra, 30 kDa cutoff) (Millipore, Billerica, Mass., USA) and filter sterilized with 0.22 μm PES filters (Thermo Scientific, Rockford, Ill., USA).

### Preparation of Anti-Phage Nanoparticles

Anti-phage SERS reporter particles were prepared by conjugation of 50-60 nm diameter SERS-5440 Nanotags (SERS NPs, OD 24) (Becton Dickinson, Research Triangle Park, NC, USA) with purified polyclonal anti-A511 antibodies. A 1 mg/mL solution of the crosslinker, sulfosuccinimidyl 4-[N-maleimidomethyl] cyclohexane-1-carboxylate (sulfo-SMCC) (Thermo Scientfic, Rockford, Ill.) was prepared in degassed conjugation buffer, 10 mM 3-morpholinopropane-1-sulfonic acid (MOPS) (Sigma Aldrich, St. Louis, Mo., USA) (pH 7.2). Next, 1.17 uL of the sulfo-SMCC was reacted with 8.55 uL of purified anti-A511 antibodies (1 mg/mL) (50 molar excess sulfo-SMCC to antibody) for 30 min at 23° C. Antibody-crosslinker complexes were reacted with SERS NPs (350 molar excess antibodies to SERS NPs) by mixing 200 μL of conjugation buffer and 200 μL SERS NPs (600 pM), followed by addition of the antibody-crosslinker complex, and reacted with continuous inversion at 23° C. for 3 h. A 1 mg/mL solution of N-ethylmaleimide (NEM) (Thermo Scientific, Rockford, Ill., USA) was prepared in degassed conjugation buffer and used as a blocker for unreacted, free thiols; 12.43 μL of this solution was added (650,000 molar excess NEM to SERS NPs). Concomitantly, 40 μL of Blocker™ Casein in PBS (Thermo Scientific, Rockford, Ill., USA) was added to block the surface of the SERS NPs. Blocking was performed at 23° C. for 2 h with continuous inversion. Unreacted sulfo-SMCC malimide groups were quenched with 40 μL of 10 mg/mL 2-mercaptoethanesulfonic acid (MESA) (MP Biomedicals, Santa Ana, Calif., USA) at 23° C. with continuous inversion for 45 min. Excess reagents were removed by centrifugation (1000×g for 10 min) and the supernatant removed and replaced with 200 μL of storage buffer, 50 mM sodium borate (Fisher Scientific, Fairlawn, N.J., USA), 1% v v-1 gelatin (telostean gelatin from cold water fish skin, Sigma Aldrich, St. Louis, Mo., USA), 0.05% w v-1 sodium azide (Fisher Scientific, Fairlawn, N.J., USA) (pH 7.5). This was repeated 4 times and conjugated particles were stored at 4° C. Fig. S3 (Supplementary materials) shows a schematic overview of the conjugation.

### LFI Device Fabrication.

Nitrocellulose membranes (Millipore Hi-Flow 180, Billerica, Mass., USA, SHF1800425) were prepared by applying purified anti-A511 antibodies as a test line and NeutrAvidin™ Biotin-Binding Protein (Thermo Scientific, Rockford, Ill.) as a control line using an IVEK Digispense 2000 striper (IVEK Corporation, North Springfield, Vt.). Anti-A511 (2 mg/mL) and NeutrAvidin (1.25 mg/mL) were applied at a rate of 4 μL/s and dried for 15 min at 35° C. Antibody/control line-stripped membranes were stored desiccated at 23° C. Release pads (Schleicher & Schuell, Keene, N.H.) were prepared by impregnation of glass fiber release medium with a solution of SERS reporter nanoparticles (0.02% solids) and control particles (0.01% solids) in 2 mM sodium borate, 0.1 M NaCl, 1% v/v fish gelatin, and 0.05% w v-1 sodium azide, 3% w/v sucrose (Baker, Phillipsburg, N.J.) (pH 8.4) dried for 30 min at 35° C. and stored desiccated at 23° C. until assembly.

LFI devices were fabricated by mating sample pad, release pad, nitrocellulose membrane, and absorbing pad (Schleicher & Schuell, Keene, N.H.) to an adhesive backboard (G&L Precision Die Casting, San Jose, Calif.) with an overlap of approximately 2 mm between layers (FIG. 1). Assembled LFI strips were cut to a width of 3.7 mm using a programmable sheer (Kinematic Automation Inc., Twain Harte, Calif.) and desiccated at 23° C. until use.

### Results

Previous conjugation methods used a phosphate-based solution, specifically, 50 mM phosphate (pH 7.15). However, use of this phosphate solution resulted in aggregation of NPs during conjugation. Alternative buffers were investigated to address NP aggregation during conjugation. The present experiments identified a sulfonic acid-based buffer, 3-(N-morpholino)propanesulfonic acid (MOPS), as being able to minimize aggregation (see FIG. 1C). Specifically, in these experiments 10 mM MOPS (pH 7.15) buffer was used during conjugation to minimize agglomeration.

Precise antibody:SERS NP molar ratios were also tested for their effect on NP agglomeration. It was determined that high antibody:NP molar ratios (for example, greater than about 350:1) led to the development of a signal in phage-free controls, with higher ratios (e.g. above 700:1) giving rise to a visually observable false positive. Although molar ratios below 350:1 resulted in decreased sensitivity, these ratios helped to minimize false positives. Blocking of free, reactive thiols on NPs further helped reduce NP agglomeration and particle precipitation during conjugation. Specifically, N-Ethylmaleimide (NEM), a maleic acid derivative having an imide ring, was used to block remaining unreacted thiols after conjugation.

Optimization of the running buffer composition was also performed. Running buffer was used to aid in solubilizing reporter and control particles deposited in the LFI's release pad. Running buffer also served as a mobile phase for transporting the particles down the membrane to the test strips. Initially, a buffer comprising sulfonic acid and a surfactant was used, 50 mM HEPES, 0.05% Tween (pH 8), however, this buffer failed to transport the NPs consistently and evenly across the LFI membrane to the absorbing pad. Specifically, the HEPES/Tween buffer resulted in a heterogeneous distribution of particles throughout the membrane. Experiments indicated that a buffer having higher concentrations of surfactant, and based on borate, rather than sulfonic acid led to improved results. Specifically, a buffer comprising 0.1 M sodium borate, 1% Tween 20, pH 8 resulted in minimizing non-specific binding, allowing the particles to transit the membranes to the wicking pads.

Other factors were optimized, including capture-antibody concentration, membrane flow rate, and NP release-pad concentration. Capture antibody application concentration was optimal at 2 mg/mL, because concentrations higher than 2 mg/mL resulted in lower sensitivity. Capillary flow rate of 180 sec 4 mm⁻¹ was optimal, because rates of 120 or 90 sec 4 mm⁻¹ resulted in decreased sensitivity. A final concentration of 0.02% solids SERS NPs at the release-pad was determined to be optimal, because higher particle concentrations caused an increase in false-positives, while lower concentrations led to decreased sensitivity.

### Experiment II—Determination of LFI Limit of Detection (LOD).

Visual and spectroscopic limits of detection of phage using the disclosed nanoparticles and LFI devices were investigated.

### Determination of LFI Limit of Detection.

LFI LOD was determined by serial dilution of filter sterilized A511 in tryptose and 1 mM CaCl₂. Triplicate analysis of phage dilutions ranging from 1.46×10⁹ plaque forming units (pfu) mL⁻¹ to 2.33×10⁰ pfu mL⁻¹ were tested, in addition to phage-free controls. Prior to application, phage samples were mixed with running buffer at a 1:1 ratio to a volume of 100 μL and applied drop wise to the sample pad. The running buffer consisted of 0.1 M sodium borate, 3% w/v bovine serum albumin (BSA) (Sigma Aldrich, St. Louis, Mo.), 1% v/v Tween®20 (Sigma Aldrich, St. Louis, Mo.) (pH 8). LFI was conducted for 30 min, wicking pads were removed to prevent backflow and LFI strips were dried in a desiccator for 10 min. Test lines were interrogated by Raman spectroscopy (Advantage 785, DeltaNu Inc., Laramie, Wyo.) at 785 nm with a laser power of 51 mW. Twelve measurements were collected along the test line with an interrogation time of 3 sec per measurement. Whole spectra analyses of SERS samples were performed by assuming sample spectra were linear combinations of a SERS nanoparticle reference spectrum and a nitrocellulose reference spectrum and solved using least squares.

### Results

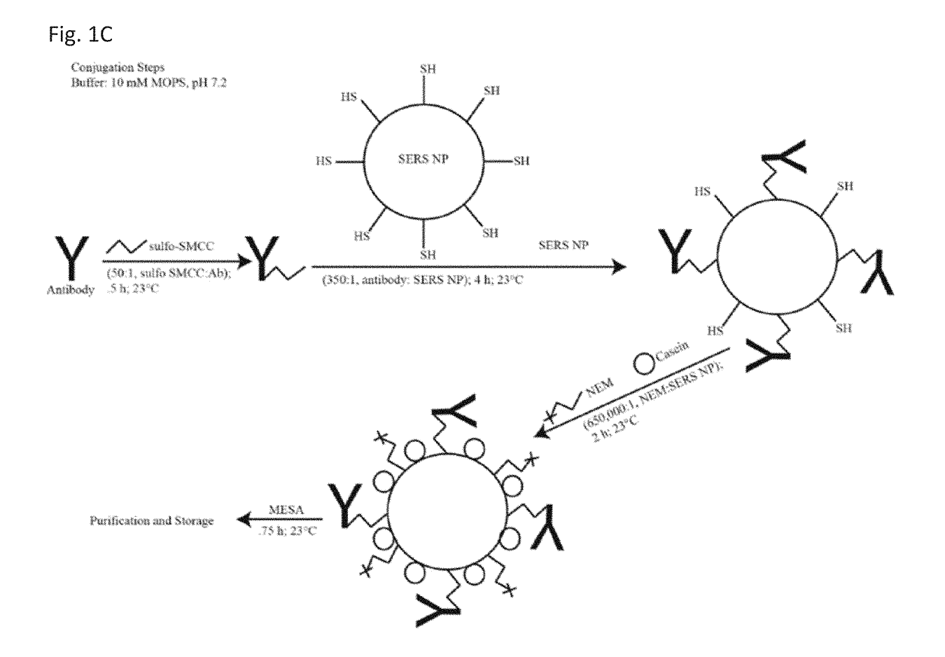

As shown in FIG. 2, positives were visually determined by formation of a pink-colored test line. In these experiments, phage concentrations of 1.46×10⁹ pfu mL⁻¹ and 1.78×10⁸ pfu mL⁻¹ produced visible pink lines. A colored line could be visually detected down to 6.10×10⁷ pfu mL⁻¹. However the line was very faint at 10⁷ pfu/mL, which could result in being graded as a negative result.

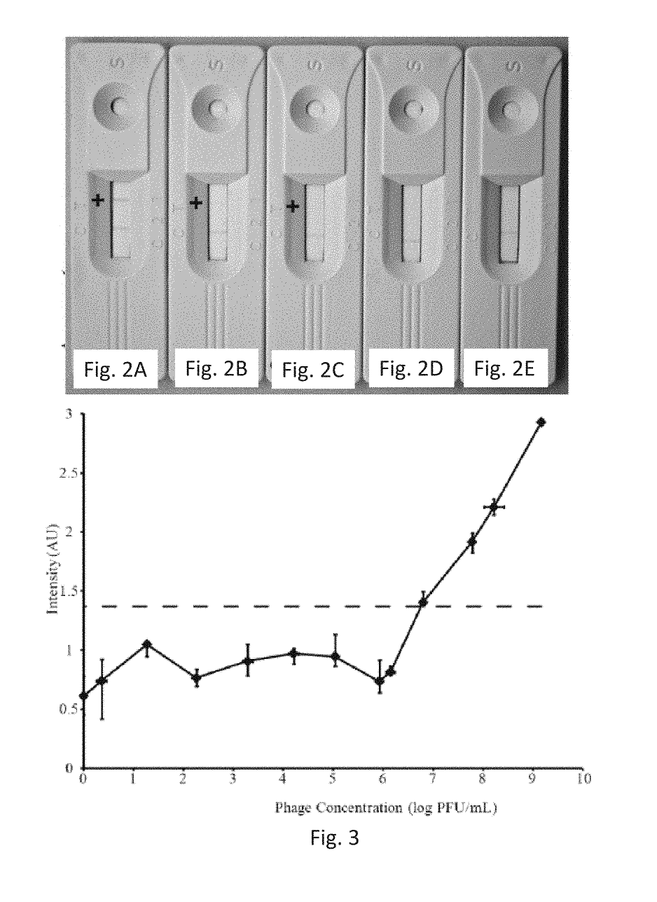

Serial dilutions of phage A511 from about 1-10 log PFU/mL were analyzed by LFI-SERS. An interquartile range (IQR) for each test line was determined by sampling 12 positions along the length of the test line. IQR was necessary because of a heterogeneous pore distribution in the membrane. The observed pore-distribution heterogeneity led to heterogeneity of Raman intensity along the length of the test line. FIG. 3 is a graph of Phage concentration vs. Raman intensity for the various phage titers. A dashed line indicates the limit of detection by SERS.

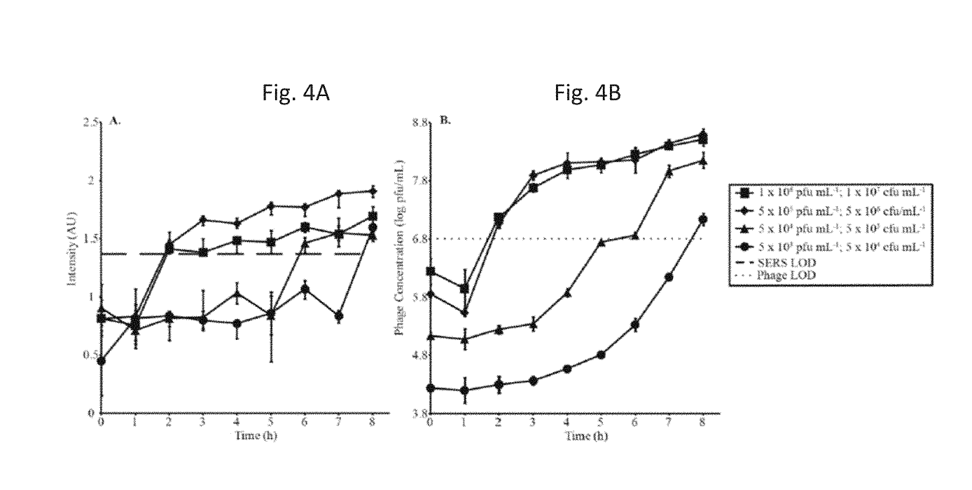

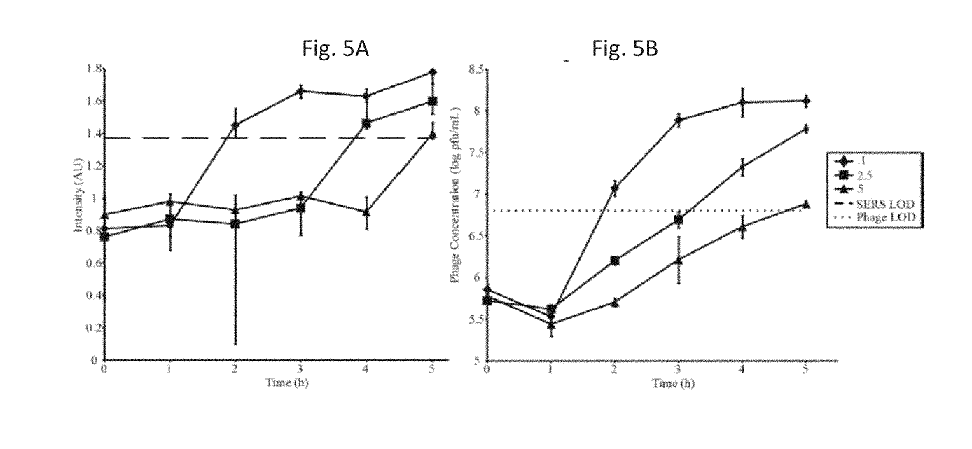

In these experiments, the lowest phage dilution to produce a Raman signal above that seen with phage-free controls was 6.37×10⁶ pfu mL⁻¹. Thus, this phage dilution (represented by a dotted line in FIGS. 4B. and 5B) and resulting Raman signal intensity of 1.37 (represented by a dashed line in FIGS. 4A and 5B) were identified as the phage and SERS LOD, respectively. A comparison between SERS and visual limits of detection (6.37×10⁶ pfu mL⁻¹ and 6.10×10⁷ pfu mL⁻¹, respectively) demonstrated an order of magnitude greater sensitivity for SERS over visual detection. All IQR measurements below SERS LOD was attributed to instrument noise.

### Experiment III—Phage Amplification and SERS-LFI Analysis.

Four phage amplification experiments were performed using A511 and L. monocytogenes ATCC 19115 at a multiplicity of infection (MOI) of 0.1 with varying A511 starting concentrations (1×10⁶, 5×10⁵, 5×10⁴, and 5×10³ pfu mL⁻¹). Two additional phage amplification experiments were conducted at MOIs of 2.5 and 5 with the same starting phage concentration (5×10⁵ pfu mL⁻¹). A511 starting concentration was always below the determined phage LOD to avoid false positive results caused by initial infecting phage. Overnight cultures of L. monocytogenes were back diluted and grown to an OD₆₂₀ of 0.3, corresponding to 1.0×10⁸ colony forming units (cfu) mL⁻¹, then diluted to the appropriate starting concentration. A511 (5×10⁷ pfu mL⁻¹) was added at appropriate volumes for the desired starting phage concentrations. Aliquots were taken from amplification reactions every hour and filtered to remove bacteria. Fifty microliters of resulting filtrates were mixed with 50 μL running buffer and applied to LFI strips as described in the previous section. Time was required to run the LFI device (˜30 min), dry the strip (˜10 min) and interrogate the LFI strip with the Raman spectrometer (˜5 min). Phage concentration was also followed in parallel for the duration of the experiment by spot titer assay as previously described to confirm results of the SERS-LFI.

### Results

Four phage amplification experiments at a MOI of 0.1 were analyzed by SERS-LFI (FIG. 4A, Table 1) and parallel spot titer assay (FIG. 4B). Phage concentrations were varied from 1×10⁶ pfu mL⁻¹ to 5×10³ pfu mL⁻¹ and corresponding bacterial concentrations were varied from 1×10⁷ cfu mL⁻¹ to 5×10⁴ cfu mL⁻¹. SERS detection of the highest phage concentrations (1×10⁶ pfu mL⁻¹ and 5×10⁵ pfu mL⁻¹) was achieved in 2 h. Additional time was required before decreased phage concentrations reached detectable levels (6 hours for 5×10⁴ pfu mL⁻¹ and 8 h for 5×10³ pfu mL⁻¹). Parallel spot titer assays confirmed that all positive tests represented phage concentrations greater than the established LOD and that increased SERS intensities correlated with phage amplification.

TABLE 1

| | | | |
|---|---|---|---|
| Phage concentration | Bacterial concentration | | SERS-LFI |
| (pfu mL⁻¹) | (cfu mL⁻¹) | MOI | Detection Time (h) |

Phage Amplification Series 1

| | | | |
|---|---|---|---|
| 1 × 10⁶ | 1 × 10⁷ | 0.1 | 2 |
| 5 × 10⁵ | 5 × 10⁶ | 0.1 | 2 |
| 5 × 10⁴ | 5 × 10⁵ | 0.1 | 6 |
| 5 × 10³ | 5 × 10⁴ | 0.1 | 8 |

Phage Amplification Series 2

| | | | |
|---|---|---|---|
| 5 × 10⁵ | 1 × 10⁶ | 0.1 | 2 |
| 5 × 10⁵ | 2 × 10⁵ | 2.5 | 4 |
| 5 × 10⁵ | 1 × 10⁵ | 5 | 5 |

A second series of amplifications were conducted to investigate the relationship between MOI and detection time (FIG. 5, Table 1). As discussed above, an MOI of 0.1 resulted in detection in 2 h. Detection times increased with increasing MOIs. MOIs of 2.5 and 5 surpassed detection limits at 4 h and 5 h, respectively. Parallel spot titer assays of (FIG. 5B). Error bars in FIGS. 4A and 5A correspond to the IQR of 12 random shots along the test line of a single LFI strip, while error bars in FIGS. 4B and 5B represented standard deviation of phage titers measured in triplicate.

Disclosed herein is the first report of phage amplification combined with the use of SERS and LFI for detection of Listeria. The disclosed experiments were directed to creating a robust anti-phage conjugation protocol and on optimization of LFI construction, with the goal of minimized nanoparticle agglomeration and improved reproducibility. The resulting devices are capable of detecting progeny A511 at concentrations as low as 6.37×10⁶ pfu mL⁻¹. The shortest detection time for L. monocytogenes was 2 h, while in a separate experiment detection of bacteria at a concentration of 1×10⁴ cfu mL⁻¹ was achieved in 8 h. While an enrichment step aid in creating bacterial concentrations that allow for propagation of progeny phage to detectable levels, phage amplification eliminates the need for downstream plating on selective media, or further biochemical or molecular tests (reducing detection by 24-48 h), while providing evidence of viable cells. SERS-LFI allows for positive identification in as little as 30 min, rather than a traditional plaque assay's 24 h.

Although the invention has been described with reference to certain embodiments, persons skilled in the art will recognize that changes can be made in form and detail without departing from the spirit and scope of the subject matter disclosed herein.

All references disclosed herein are incorporated by reference as if the disclosure of each was expressly disclosed.

What is claimed is:

1. A Listeria phage-specific antibody-presenting particle comprising:
a gold metal core;
a coating comprising at least one Raman active reporter molecule attached to the surface of the metal core;
a glass coating surrounding said coating; and
a phage-specific antibody attached to the surface of said glass coating.

2. The antibody-presenting particle of claim 1, wherein the Raman active reporter is trans-1,2-bis(4-pyridyl)-ethylene.

3. The antibody-presenting particle of claim 2, wherein the glass coating comprises SiO functionalized with thiol groups.

4. The antibody-presenting particle of claim 1, further comprising a metal shell overlaying the core.

5. The antibody-presenting particle of claim 4, wherein the metal shell is comprised of Au/AuS.

6. The antibody-presenting particle of claim 1, wherein the core is a sphere with a diameter between 15-200 nm.

7. The antibody-presenting particle of claim 6, wherein the core has a diameter of about 50-60 nm.

8. The antibody-presenting particle of claim 1, wherein the phage specific antibody recognizes Listeria phage A511.

9. The antibody-presenting particle of claim 8, wherein the phage specific antibody is polyclonal

10. The antibody-presenting particle of claim 8, wherein the phage specific antibody is monoclonal.

11. A method of creating a Listeria phage-specific antibody-presenting nanoparticle the method comprising:
combining a sulfosuccinimidyl crosslinker with a sulfonic acid buffer to create a crosslinker mixture;
combining the Listeria phage specific antibody with the crosslinker mixture to create an antibody mixture;
combining the antibody mixture with the nanoparticle, where in the molar ratio of antibody to nanoparticles is about 350:1 to create a conjugated nanoparticle, and
thereby creating a Listeria phage-specific antibody-presenting nanoparticle.

12. The method of claim 11, wherein the sulfosuccinimidyl crosslinker is sulfosuccinimidyl 4-[N-maleimidomethyl] cyclohexane-1-carboxylate (sulfo-SMCC).

13. The method of claim 12, wherein the sulphonic acid is 3-morpholinopropane-1-sulfonic acid.

14. The method of claim 13, wherein the step of combining the Listeria antibody and crosslinker a 50 molar excess sulfo-SMCC to antibody is used.

15. The method of claim 14, further comprising a step of reacting unreacted thiols, wherein N-ethylmaleimide is combined with the conjugated nanoparticle.

16. The method of claim 11, wherein the core is a sphere with a diameter between 15-200 nm.

17. The method of claim 16, wherein the core has a diameter of about 50-60 nm.

18. The method of claim 11, wherein the phage specific antibody recognizes Listeria phage A511.

19. The method of claim 18, wherein the phage specific antibody is polyclonal

20. The method of claim 18, wherein the phage specific antibody is monoclonal.
